(19) United States
(12) Patent Application Publication
(10) Pub. No.: US 2019/0246876 A1
(43) Pub. Date: Aug. 15, 2019

(54) COMPOSITIONS AND METHODS FOR DIRECTING ENDOSCOPIC DEVICES

(71) Applicant: NeuWave Medical, Inc., Madison, WI (US)

(72) Inventor: Matthew Schaning, Madison, WI (US)

(21) Appl. No.: 16/266,811

(22) Filed: Feb. 4, 2019

Related U.S. Application Data
(60) Provisional application No. 62/631,152, filed on Feb. 15, 2018.

(51) Int. Cl. A61B 1/005; A61B 18/18; A61B 1/01; A61B 1/00; A61B 1/267

(52) U.S. Cl. CPC A61B 1/0051; A61B 18/1815; A61B 1/01; A61B 2018/00577; A61B 1/00087; A61B 1/00133; A61B 1/2676; A61B 1/00066

(57) ABSTRACT

The present invention relates to comprehensive systems, devices and methods for directing endoscopy devices. In particular, provided herein are endoscopy directing devices and uses thereof. The devices described herein find use in a variety of endoscopy (e.g., bronchoscopy) applications.

### FIELD OF THE INVENTION

The present invention relates to comprehensive systems, devices and methods for directing endoscopy devices. In particular, provided herein are endoscopy directing devices and uses thereof. The devices described herein find use in a variety of endoscopy (e.g., bronchoscopy) applications.

### BACKGROUND

Ablation is an important therapeutic strategy for treating certain tissues such as benign and malignant tumors, cardiac arrhythmias, cardiac dysrhythmias and tachycardia. Most approved ablation systems utilize radio frequency (RF) energy as the ablating energy source. Accordingly, a variety of RF based catheters and power supplies are currently available to physicians. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation to form on the energy emitting electrodes which limits the further deposition of electrical energy.

Microwave energy is an effective energy source for heating biological tissues and is used in such applications as, for example, cancer treatment and preheating of blood prior to infusions. Accordingly, in view of the drawbacks of the traditional ablation techniques, there has recently been a great deal of interest in using microwave energy as an ablation energy source. The advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures. Accordingly, there are a number of devices under development that utilize electromagnetic energy in the microwave frequency range as the ablation energy source (see, e.g., U.S. Pat. Nos. 4,641,649, 5,246,438, 5,405,346, 5,314,466, 5,800,494, 5,957,969, 6,471,696, 6,878,147, and 6,962,586; each of which is herein incorporated by reference in their entireties).

Unfortunately, current devices are limited, by size and flexibility, as to the body regions to which they are capable of delivering energy. For example, in the lungs, the air paths of the bronchial tree get progressively narrower as they branch with increasing depth into the periphery of the lungs. Accurate placement of energy delivery devices to such difficult to reach regions is not feasible with current devices.

Improved systems and devices for delivering energy to difficult to reach tissue regions are needed.

The present invention addresses such needs.

### SUMMARY OF THE INVENTION

Imprecise movement and poor tactile and/or quantitative feedback of endoscopic tools (e.g., microwave ablation devices) is an impediment to their precise function, especially in difficult to reach areas. As such, more precise and controlled manipulation of such endoscopic tools would be beneficial in treatment. Typically, manipulation of such endoscopic tools is manual, using imaging and/or the tactile feel of the tool during insertion for advancing. Imaging is used to confirm tip displacement distance. Such existing manual methods are adequate, but not exceptional, as doctors are often questioning exactly where the tip of a tool is and examining images for confirmation.

The more precise the endoscopic tool advancement is and the better insertion depth feedback that is used, the better the treatment results.

Accordingly, provided herein are improved devices, systems and methods for advancing and directing endoscopy tools (e.g., microwave ablation devices). Indeed, the devices described herein provide improved manual and automatic control of endoscopy tools and, in some embodiments, provide real time feedback of the location of such tools.

In certain embodiments, the present invention provides endoscopy directing devices comprising an endoscopy tool opening, an endoscopy tool movement component, and an endoscopy tool attachment component. In some embodiments, the endoscopy tool movement component is positioned above the endoscopy tool attachment component. In some embodiments, the endoscopy tool opening is a hollow channel that extends through the endoscopy tool movement component and the endoscopy tool attachment component. In some embodiments, the endoscopy tool movement component is configured to incrementally move an endoscopy tool positioned within the endoscopy tool opening. In some embodiments, the endoscopy tool attachment component is configured to secure with an endoscopy tool port. In some embodiments, the width of the endoscopy tool opening is between 2 and 4 mm. In some embodiments, the endoscopy tool movement component comprises two or more rotating wheels designed to simultaneously engage with an endoscopy tool positioned within the endoscopy tool opening such that rotation of such rotating wheels results in the incremental movement of the endoscopy tool. In some embodiments, the rotation of the two or more rotating wheels is manual or automatic. In some embodiments, the amount of incremental movement is between 1 and 2 mm. In some embodiments, the endoscopy tool attachment component is configured to secure with an endoscopy tool port. In some embodiments, the endoscopy tool is a microwave ablation device.

In certain embodiments, the present invention provides systems comprising an endoscopy directing device (described above), an endoscope, wherein the endoscopy tool attachment component is engaged with an endoscopy tool port of said endoscope. In some embodiments, the endoscope is a bronchoscope. In some embodiments, the system further comprises an endoscopy tool. In some embodiments, the endoscopy tool is positioned in the endoscopy tool opening of said device. In some embodiments, the endoscopy tool is a biopsy tool. In some embodiments, the endoscopy tool is an ablation tool. In some embodiments, the ablation tool is a microwave ablation device. In some embodiments, the system further includes a processor for operation of the components of said system.

In certain embodiments, the present invention provides methods for directing an endoscopy tool, comprising a) providing an endoscopy tool, an endoscope, and an endoscope directing device as described herein, b) securing the endoscopy directing device with the endoscopy tool port, c) positioning the endoscopy tool through the endoscopy tool opening such that the rotational wheels are in contact with the endoscopy tool, d) directing the endoscopy tool to a preferred location through rotation of the rotational wheels. In some embodiments, the endoscopy tool is located in a lung of a subject. In some embodiments, the endoscopy tool is a microwave ablation device. In some embodiments, the endoscopy directing device is configured to direct the positioning of an endoscopy tool (e.g., microwave ablation device) located in the lung of a subject.

Additional embodiments are described herein.

### BRIEF DESCRIPTION OF THE DRAWINGS

FIG. 1 shows an exemplary endoscopy directing device engaged with an endoscopy tool and an endoscopy tool port.

FIG. 2 shows an alternate view of an endoscopy device engaged with an endoscopy tool port and an endoscopy tool.

### DETAILED DESCRIPTION

The present invention relates to comprehensive systems, devices and methods for directing endoscopy devices. In particular, provided herein are endoscopy directing devices and uses thereof. The devices described herein find use in a variety of endoscopy (e.g., bronchoscopy) applications. Examples include, but are not limited to, obtaining biopsies and delivering energy to tissue for a wide variety of applications, including medical procedures (e.g., tissue ablation, resection, cautery, electrosurgery, tissue harvest, etc.).

In particular, systems, devices, and methods are provided for treating a difficult to access tissue region (e.g., a peripheral lung tumor) through use of the systems of the present invention.

The endoscopy locating and directing systems of the present invention may be combined within various system/kit embodiments. For example, the present invention provides systems comprising one or more of a generator, a power distribution system, a means of directing, controlling and delivering power (e.g., a power splitter), an energy applicator, device placement systems (e.g. multiple catheter system), along with any one or more accessory component (e.g., surgical instruments, software for assisting in procedure, processors, temperature monitoring devices, etc.). The present invention is not limited to any particular accessory component.

The systems of the present invention may be used in any medical procedure (e.g., percutaneous or surgical) involving delivery of energy (e.g., radiofrequency energy, microwave energy, laser, focused ultrasound, etc.) to a tissue region. The systems are not limited to treating a particular type or kind of tissue region (e.g., brain, liver, heart, blood vessels, foot, lung, bone, etc.). For example, the systems of the present invention find use in ablating tumor regions (e.g. lung tumors (e.g. peripheral lung tumors)). Additional treatments include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, sterilization procedures (e.g., ablation of the fallopian tubes) and cauterization of blood vessels or tissue for any purposes. In some embodiments, the surgical application comprises ablation therapy (e.g., to achieve coagulative necrosis). In some embodiments, the surgical application comprises tumor ablation to target, for example, primary or metastatic tumors or peripheral lung nodules. In some embodiments, the surgical application comprises the control of hemorrhage (e.g. electrocautery). In some embodiments, the surgical application comprises tissue cutting or removal. In some embodiments, the device is configured for movement and positioning, with minimal damage to the tissue or organism, at any desired location, including but not limited to, the brain, neck, chest, abdomen, pelvis, and extremities. In some embodiments, the device is configured for guided delivery, for example, by computerized tomography, ultrasound, magnetic resonance imaging, fluoroscopy, and the like.

The illustrated embodiments provided below describe the devices and systems of the present invention in terms of medical applications (e.g., endoscopic uses for ablation of tissue through delivery of microwave energy). However, it should be appreciated that the systems of the present invention are not limited to energy delivery applications. The systems may be used in any setting requiring endoscopy (e.g., biopsy or imaging) and for delivery of energy to a load (e.g., agricultural settings, manufacture settings, research settings, etc.). The illustrated embodiments describe the systems of the present invention in terms of microwave energy.

Provided herein are improved devices, systems and methods for advancing and directing endoscopy tools (e.g., microwave ablation devices). Indeed, the devices described herein provide improved manual and automatic control of endoscopy tools and, in some embodiments, provide real time feedback of the location of such tools.

Such devices are not limited to a particular configuration or design. In some embodiments, such devices comprise or consist essentially of at least one of an endoscopy tool opening, an endoscopy tool movement component, and an endoscopy tool attachment component.

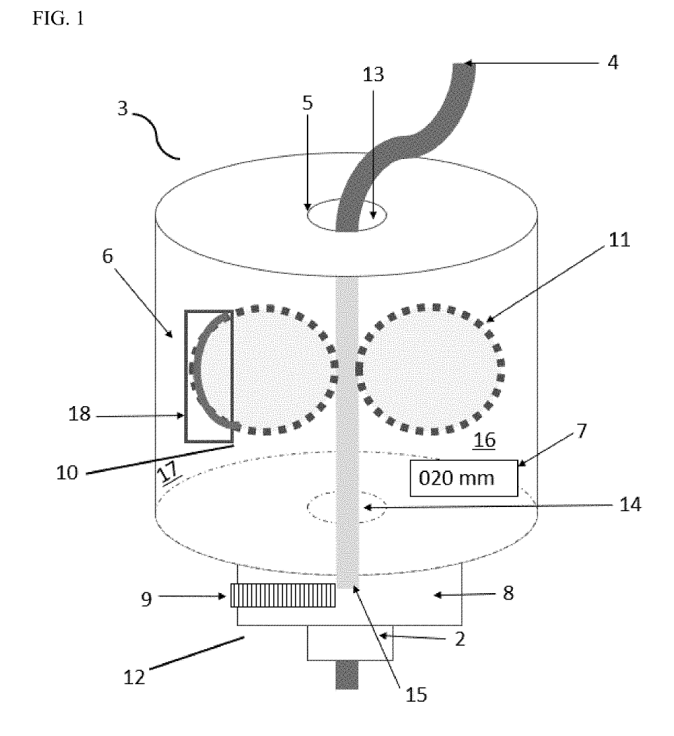

FIG. 1 shows an exemplary endoscopy directing device 3 engaged with an endoscopy tool 4 and an endoscopy tool port 2. Such endoscopy directing devices 3 are not limited to a particular manner of engagement with an endoscopy tool 4 and endoscopy tool port 2 (described in more detail below).

Still referring to FIG. 1, the endoscopy directing device 3 has an endoscopy tool opening 5, an endoscopy tool movement component 11, and an endoscopy tool attachment component 12. The endoscopy directing device 3 is not limited to specific configurations and/or designs for the endoscopy tool opening 5, the endoscopy tool movement component 11, and the endoscopy tool attachment component 12. In some embodiments, the aspects and configurations for the endoscopy tool opening 5, the endoscopy tool movement component 11, and the endoscopy tool attachment component 12 render the endoscopy directing device 3 capable of improved manual and automatic control of endoscopy tools and, in some embodiments, real time feedback of the location of such tools.

Still referring to FIG. 1, the endoscopy directing device 3 is not limited to specific configurations and/or designs for the endoscopy tool opening 5. In some embodiments, as shown, the endoscopy tool opening 5 is an opening that extends through the entirety of the endoscopy directing device 3 essentially rendering it a hollow channel capable of engaging with an outside component (e.g., endoscopy tool 4 and/or endoscopy tool port 2) (described in more detail below).

Indeed, in some embodiments, as shown in FIG. 1, the endoscopy tool opening 5 defines a central axis through the endoscopy directing device 3. As shown in FIG. 1, the endoscopy tool opening 5 extends through the entirety of both the endoscopy tool movement component 11 and the endoscopy tool attachment component 12. As shown, the endoscopy tool opening 5 has a top opening 13 positioned at the top of the endoscopy tool movement component 11, a mid-portion opening 14 positioned at the junction of the endoscopy tool movement component 11 and the endoscopy tool attachment component 12, and a bottom opening 15 positioned at the bottom of the endoscopy tool attachment component 12.

The endoscopy tool opening 5 is not limited to a particular width and/or length. In some embodiments, the width of the endoscopy tool opening 5 is In some embodiments, the width of the endoscopy tool opening 5 is between approximately 0.5 mm and 7 mm (e.g., 0.75 mm and 6 mm; 1 mm and 5 mm; 2 mm and 4 mm; 2.5 mm and 3.5 mm; 2.8 mm and 3.2 mm; 2.95 mm and 3.1 mm; 2.99 mm and 3.01 mm). As shown in FIG. 1, the width of the endoscopy tool opening 5 is 3 mm. In some embodiments, the width is consistent throughout the entirety of the endoscopy tool opening 5. In some embodiments, the width is inconsistent throughout the entirety of the endoscopy tool opening 5 (e.g., larger at the top and/or bottom of the endoscopy tool opening 5). In some embodiments, as shown in FIG. 1, the width is consistent throughout the entirety of the endoscopy tool opening 5. In some embodiments, as shown in FIG. 1, the length of the endoscopy tool opening 5 extends through the entirety of the endoscopy directing device 3. In some embodiments, the width and/or length of the endoscopy tool opening 5 is such that an endoscopy tool 4 is capable of being directed through the top opening 13, through the mid portion, and out through the bottom opening 15.

Still referring to FIG. 1, the endoscopy directing device 3 is not limited to a specific shape for the endoscopy tool opening 5. In some embodiments, as shown, the shape of the endoscopy tool opening 5 is circular through its entirety. In some embodiments, the shape of the endoscopy tool opening 5 is square shaped, oval shaped, rectangular shaped, and/or any mixture of shapes. In some embodiments, the shape of the endoscopy tool opening 5 is such that an endoscopy tool 4 is capable of being directed through the top opening 13, through the mid portion opening 14, and out through the bottom opening 15 of the endoscopy tool opening 5.

Still referring to FIG. 1, the endoscopy directing device 3 is not limited to specific configurations and/or designs for the endoscopy tool movement component 11. In some embodiments, as shown, the specific configurations and/or designs for endoscopy tool movement component 11 render the endoscopy directing device 3 capable of improved manual and automatic control of endoscopy tools and, in some embodiments, real time feedback of the location of such tools.

In some embodiments, as shown in FIG. 1, the endoscopy tool movement component 11 has an endoscopy tool movement component internal region 16 and an endoscopy tool movement component external region 17. The endoscopy tool movement component 11 is not limited to a particular shape or size. In some embodiments, the shape and size of the endoscopy tool movement component 11 is such that it is able to render the endoscopy directing device 3 capable of improved manual and automatic control of endoscopy tools and, in some embodiments, real time feedback of the location of such tools.

Still referring to FIG. 1, the endoscopy tool movement component 11 is not limited to a particular manner of controlling the movement of an endoscopy tool 4. In some embodiments, as shown in FIG. 1, the endoscopy tool movement component internal region 16 has therein a plurality (e.g., 1 or 2) of rotating wheels 6 designed to engage with an endoscopy tool 4 positioned within the endoscopy tool opening 5 such that rotation of such rotating wheels 6 results in an incremental movement of the endoscopy tool 4 (described in more detail below).

Still referring to FIG. 1, the endoscopy tool movement component internal region 16 is not limited to a particular number of rotating wheels 6. In some embodiments, as shown in FIG. 1, the endoscopy tool movement component internal region 16 has therein two rotating wheels 6. In some embodiments, the endoscopy tool movement component internal region 16 has therein a plurality of rotating wheels 6 (e.g., 3, 4, 5, 6, 7, 8, 9, 10, 15, 20, 100, etc). In some embodiments, the amount of rotating wheels 6 is such that it renders the endoscopy directing device 3 capable of improved manual and automatic control of endoscopy tools and, in some embodiments, real time feedback of the location of such tools.

Still referring to FIG. 1, the endoscopy tool movement component internal region 16 is not limited to a particular size of the rotating wheels 6. In some embodiments, the size of the rotating wheels 6 is such that such rotating wheels 6 are capable of positioning and rotation within the endoscopy tool movement component internal region 16.

Still referring to FIG. 1, the endoscopy tool movement component internal region 16 is not limited to a particular positioning of the rotating wheels 6. In some embodiments, as shown in FIG. 1, each of the rotating wheels 6 are positioned opposite each other with the endoscopy tool opening 5 channel positioned between the rotating wheels 6. In some embodiments, as shown in FIG. 1, the rotating wheels 6 are further positioned to be in contact with endoscopy tool opening 5 such that an endoscopy tool 4 positioned within the endoscopy tool opening 5 would be engaged with each of the rotating wheels 6, and rotation of the rotating wheels 6 would result in movement of the endoscopy tool 4 in either a forward or reverse directing depending on the direction of rotation of the rotating wheels 6. For example, in embodiments wherein the rotating wheels 6 are positioned opposite each other and opposite an endoscopy tool 4 positioned with the endoscopy tool opening 6, the rotating wheels 6 are in engagement with such an endoscopy tool 4. Such engagement permits directional movement of the endoscopy tool 4 through rotation of one of the rotating wheels 6 which results in rotation of the second rotating wheel 6 upon movement of the endoscopy tool 4.

As such, such engagement between the rotational wheels 6 and an endoscopy tool 4 positioned within the endoscopy tool opening 5 permits incremental movement of the endoscopy tool 4 through the endoscopy tool opening 5 in either a forward motion or a reverse motion. This mechanism is not limited to a particular manner of rotating the rotation wheels 6. In some embodiments, as shown in FIG. 1, the endoscopy tool movement component external region 17 has a slot opening 18 wherein a user is able to access one of the rotating wheels 6 and able to rotate such rotating wheel 6. As such, as shown in FIG. 1, one of the rotating wheels 6 is positioned such that a portion of the rotating wheel 6 is exposed through the slot opening 18. In such embodiments, a user is able to manipulate the exposed rotating wheel 6 for purposes of rotating the exposed rotating wheel 6 and thereby rotating the oppositely positioned rotating wheel 6 and thereby movement of the endoscopy tool 4.

The endoscopy tool movement component 11 is not limited to a specific amount of movement of an endoscopy tool 4 positioned within the endoscopy directional device opening 5 through rotation of the rotation wheels 6. In some embodiments, the amount of movement can be as little as 0.01 mm.

In some embodiments as shown in FIG. 1, the endoscopy tool movement component 11 is configured for incremental movement of an endoscopy tool 4 positioned within the endoscopy directional device opening 5 through rotation of the rotation wheels 6. For example, in some embodiments, rotation of the rotational wheels 6 results in pre-defined incremental distance movements of the endoscopy tool 4. In some embodiments, the pre-defined incremental distance movement is approximately 0.1 mm (e.g., 0.01 mm, 0.05 mm, 0.1 mm, 0.25 mm, 0.35 mm, 0.5 mm, 0.75 mm, 0.8 mm, 0.95 mm, 0.99 mm, 1 mm, 1.25 mm, 1.35 mm, 1.5 mm, 1.61 mm, 1.75 mm, 1.8 mm, 1.95 mm, 1.99 mm, 2 mm, 2.01 mm, 2.1 mm, 2.25 mm, etc.). In some embodiments, the rotation of the wheels 6 features a tactile click for such a designed incremental value (e.g., 1-2 mm).

The endoscopy tool movement component 11 is not limited to a particular manner of rotating the rotational wheels 6 (for purposes of moving an endoscopy tool 4 positioned within the endoscopy tool opening 5. In some embodiments, as shown in FIG. 1, rotation of the rotational wheels 6 occurs through user manipulation (e.g., finger/thumb manipulation). In some embodiments, as shown in FIG. 1, rotation of the rotational wheels 6 occurs through user manipulation (e.g., finger/thumb manipulation) of a rotational wheel 6 exposed through the slot opening 18. In some embodiments, the outer surface of each rotating wheel 6 is comprised of a compliant material with high coefficient of friction (e.g., silicone, rubber, or thermoplastic (e.g., Santoprene)) for purposes of easing such user manipulation.

Still referring to FIG. 1, the endoscopy tool movement component 11 further comprises a rotating wheel engagement/disengagement lever 10 that controls the rotating axis position of each rotating wheel 3 such that when disengaged the outer circumference of each rotating wheel 3 moves away from the endoscopy tool opening 5 and an endoscopy tool 4 positioned within the endoscopy tool opening 5 thereby precluding operable communication with the endoscopy tool 4. In some embodiments, the rotating wheels 6 are greater than 0.084 inches away from an endoscopy tool 4 when in the disengaged position. When engaged, the outer circumference of the rotating wheels 6 return to the innermost position such that the distance between each of the rotating wheels 6 is slightly smaller than the diameter of the endoscopy tool 4 (e.g., approximately 0.068 inches) and in contact with the tool 4 so that the rotational wheels 6 are able to move the endoscopy tool 4.

In some embodiments, movement of an endoscopy tool 4 positioned within an endoscopy tool opening 5 utilizes a different mechanism than the rotational wheels 6. For example, in some embodiments, such movement is automated. In some embodiments, rotation of the rotational wheels 6 occurs automatically.

Still referring to FIG. 1, in some embodiments, the endoscopy tool movement component 11 further comprises a display 7 for displaying information regarding movement related to the endoscopy direction device 3. For example, in some embodiments, the amount of total movement (e.g., forward and/or reverse) of the endoscopy tool 3 can be shown. In some embodiments, the total depth of the endoscopy device 3 is shown. In some embodiments, the amount of incremental movement of the endoscopy device 3 is shown. In some embodiments, the display 7 is analog or digital. In some embodiments, the display 7 features a zeroing function to reset the display to zero as desired. In some embodiments, distance displayed on the display 7 is measured by mechanical and/or optical methods.

Still referring to FIG. 1, the endoscopy directing device 3 is not limited to specific configurations and/or designs for the endoscopy tool attachment component 12. In some embodiments, as shown, the specific configurations and/or designs for the endoscopy tool attachment component 12 render the endoscopy directing device 3 engaged (e.g., secured) with an endoscopy tool port 2 (thereby rendering the endoscopy directing device 3 capable of improved manual and automatic control of endoscopy tools and, in some embodiments, real time feedback of the location of such tools).

The endoscopy tool attachment component 12 is not limited to a particular manner of engaging (e.g., securing) with an endoscopy tool port 2. In some embodiments, the endoscopy tool attachment component 12 utilizes a clamping mechanism 8 (e.g., a hinged clamp mount) to secure with an endoscopy tool port 2. In some embodiments, the endoscopy tool attachment component 12 utilizes a release lever 9 for disengaging securement with the endoscopy tool port 2. In some embodiments the clamping mechanism 8 operates with the release lever 9 (e.g., to engage and/or disengage securement of the endoscopy tool attachment component 12 with an endoscopy tool port 2), although other mounting options are specifically contemplated (e.g., threaded design, luer lock, etc.).

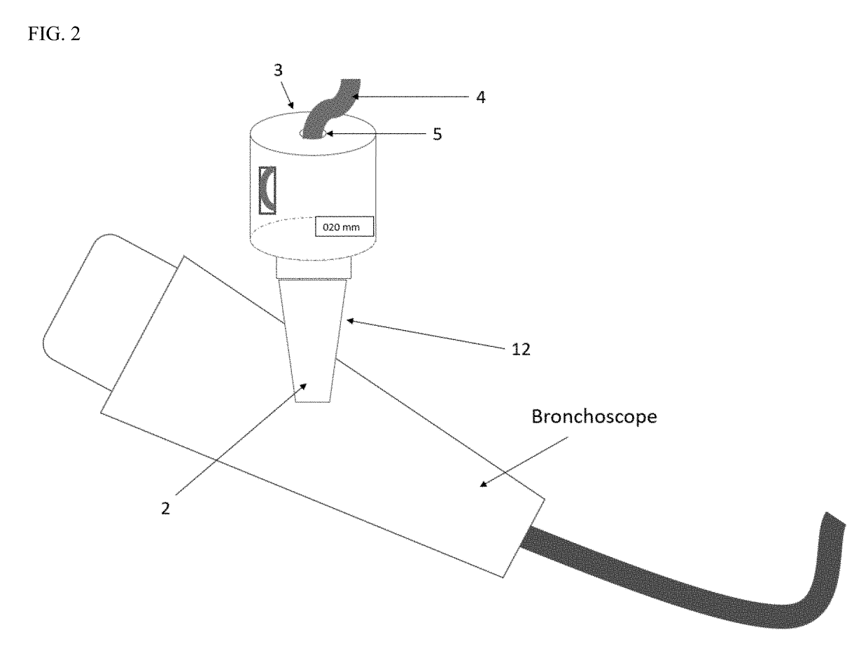

FIG. 2 shows an alternate view of an endoscopy device 3 engaged with an endoscopy tool port 2 and an endoscopy tool 4. As shown, the endoscopy tool attachment component 12 is shown engaged with the endoscopy tool port 2. As shown, an endoscopy tool 4 is shown positioned within the endoscopy tool opening 5.

In some embodiments, the endoscopy devices are permanently secured with endoscopy tool supports.

The present invention is not limited to use with particular endoscopy tools. Examples include, but are not limited to, biopsy tools and ablation tools.

In some embodiments, any suitable endoscope or bronchoscope known to those in the art finds use in the present invention. One type of conventional flexible bronchoscope is described in U.S. Pat. No. 4,880,015, herein incorporated by reference in its entirety. The bronchoscope measures 790 mm in length and has two main parts, a working head and an insertion tube. The working head contains an eyepiece; an ocular lens with a diopter adjusting ring; attachments for suction tubing, a suction valve, and light source; and an access port or biopsy inlet, through which various devices and fluids can be passed into the working channel and out the distal end of the bronchoscope. The working head is attached to the insertion tube, which typically measures 580 mm in length and 6.3 mm in diameter. The insertion tube contains fiberoptic bundles, which terminate in the objective lens at the distal tip, light guides, and a working channel. Other endoscopes and bronchoscopes which may find use in embodiments of the present invention, or portions of which may find use with the present invention, are described in U.S. Pat. Nos. 7,473,219; 6,086,529; 4,586,491; 7,263,997; 7,233,820; and 6,174,307.

In use, the endoscopy devices are mounted to an endoscope, such as a bronchoscope. The rotational wheel engagement lever is opened to disengage the wheels. After a user navigates the endoscope to the area of interest, an endoscopic tool, such as a biopsy tool or flexible ablation probe, is inserted freely through the device and the tool port. Once inserted and near the target tissue, the engagement lever is closed to engage the wheels and sandwich the tool between the compliant material on each wheel circumference. Precise insertion of the tool may then continue by rotating the finger wheel. The user receives tactile feedback from the wheel and is able to measure exactly how far the tool is being inserted using the measurement display.

As described, the devices of the present disclosure find use in a variety of endoscopy systems. In some exemplary embodiments, the system is an ablation system (See e.g., U.S. Pat. Ap. Nos. 2016/0015453 and 2013/0116679; each of which is herein incorporated by reference in its entirety).

The energy delivery systems of the present invention contemplate the use of any type of device configured to deliver (e.g., emit) energy (e.g., ablation device, surgical device, etc.) (see, e.g., U.S. Pat. Nos. 7,101,369, 7,033,352, 6,893,436, 6,878,147, 6,823,218, 6,817,999, 6,635,055, 6,471,696, 6,383,182, 6,312,427, 6,287,302, 6,277,113, 6,251,128, 6,245,062, 6,026,331, 6,016,811, 5,810,803, 5,800,494, 5,788,692, 5,405,346, 4,494,539, U.S. patent application Ser. Nos. 11/728,460, 11/728,457, 11/728,428, 11/237,136, 11/236,985, 10/980,699, 10/961,994, 10/961,761, 10/834,802, 10/370,179, 09/847,181; Great Britain Patent Application Nos. 2,406,521, 2,388,039; European Patent No. 1395190; and International Patent Application Nos. WO 06/008481, WO 06/002943, WO 05/034783, WO 04/112628, WO 04/033039, WO 04/026122, WO 03/088858, WO 03/039385 WO 95/04385; each herein incorporated by reference in their entireties). Such devices include any and all medical, veterinary, and research applications devices configured for energy emission, as well as devices used in agricultural settings, manufacturing settings, mechanical settings, or any other application where energy is to be delivered.

In some embodiments, the systems utilize energy delivery devices having therein antennae configured to emit energy (e.g., microwave energy, radiofrequency energy, radiation energy). The systems are not limited to particular types or designs of antennae (e.g., ablation device, surgical device, etc.). In some embodiments, the systems utilize energy delivery devices having linearly shaped antennae (see, e.g., U.S. Pat. Nos. 6,878,147, 4,494,539, U.S. patent application Ser. Nos. 11/728,460, 11/728,457, 11/728,428, 10/961,994, 10/961,761; and International Patent Application No., WO 03/039385; each herein incorporated by reference in their entireties). In some embodiments, the systems utilize energy delivery devices having non-linearly shaped antennae (see, e.g., U.S. Pat. Nos. 6,251,128, 6,016,811, and 5,800,494, U.S. patent application Ser. No. 09/847,181, and International Patent Application No. WO 03/088858; each herein incorporated by reference in their entireties). In some embodiments, the antennae have horn reflection components (see, e.g., U.S. Pat. Nos. 6,527,768, 6,287,302; each herein incorporated by reference in their entireties). In some embodiments, the antenna has a directional reflection shield (see, e.g., U.S. Pat. No. 6,312,427; herein incorporated by reference in its entirety). In some embodiments, the antenna has therein a securing component so as to secure the energy delivery device within a particular tissue region (see, e.g., U.S. Pat. Nos. 6,364,876, and 5,741,249; each herein incorporated by reference in their entireties).

In some embodiments, antennae configured to emit energy comprise coaxial transmission lines. The devices are not limited to particular configurations of coaxial transmission lines. Examples of coaxial transmission lines include, but are not limited to, coaxial transmission lines developed by Pasternack, Micro-coax, and SRC Cables. In some embodiments, the coaxial transmission line has a center conductor, a dielectric element, and an outer conductor (e.g., outer shield). In some embodiments, the systems utilize antennae having flexible coaxial transmission lines (e.g., for purposes of positioning around, for example, pulmonary veins or through tubular structures) (see, e.g., U.S. Pat. Nos. 7,033,352, 6,893,436, 6,817,999, 6,251,128, 5,810,803, 5,800,494; each herein incorporated by reference in their entireties). In some embodiments, the systems utilize antennae having rigid coaxial transmission lines (see, e.g., U.S. Pat. No. 6,878,147, U.S. patent application Ser. Nos. 10/961,994, 10/961,761, and International Patent Application No. WO 03/039385; each herein incorporated by reference in their entireties).

In some embodiments, the energy delivery devices have a triaxial transmission line. In some embodiments, the present invention provides a triaxial microwave probe design where the outer conductor allows improved tuning of the antenna to reduce reflected energy through the transmission line. This improved tuning reduces heating of the transmission line allowing more power to be applied to the tissue and/or a smaller transmission line (e.g. narrower) to be used. Further, the outer conductor may slide with respect to the inner conductors to permit adjustment of the tuning to correct for effects of the tissue on the tuning. In some embodiments, and outer conductor is stationary with respect to the inner conductors. In some embodiments, the present invention provides a probe having a first conductor and a tubular second conductor coaxially around the first conductor but insulated therefrom (e.g. insulated by a dielectric material and/or coolant). A tubular third conductor is fit coaxially around the first and second conductors. The first conductor may extend beyond the second conductor into tissue when a proximal end of the probe is inserted into a body. The second conductor may extend beyond the third conductor into the tissue to provide improved tuning of the probe limiting power dissipated in the probe outside of the exposed portions of the first and second conductors. The third tubular conductor may be a channel catheter for insertion into the body or may be separate from a channel catheter. In some embodiments, a device comprising first, second, and third conductors is sufficiently flexible to navigate a winding path (e.g. through a branched structure within a subject (e.g. through the brachial tree)). In some embodiments, the first and second conductors may fit slidably within the third conductor. In some embodiments, the present invention provides a probe that facilitates tuning of the probe in tissue by sliding the first and second conductors inside of the third conductor. In some embodiments, the probe includes a lock attached to the third conductor to adjustably lock a sliding location of the first and second conductors with respect to the third conductor. In some embodiments, the present invention provides a triaxial transmission line, as described in U.S. Pat. No. 7,101,369, U.S. Pat. App. No. 2007/0016180, U.S. Pat. App. No. 2008/0033424, U.S. Pat. App. No. 20100045558, U.S. Pat. App. No. 20100045559, herein incorporated by reference in their entireties.

In some embodiments, the energy delivery systems of the present invention utilize devices configured for delivery of microwave energy with an optimized characteristic impedance (see, e.g., U.S. patent application Ser. No. 11/728,428; herein incorporated by reference in its entirety).

In some embodiments, the energy delivery systems of the present invention utilize energy delivery devices having coolant passage channels (see, e.g., U.S. Pat. No. 6,461,351, and U.S. patent application Ser. No. 11/728,460; herein incorporated by reference in its entirety).

In some embodiments, the energy delivery systems of the present invention utilize energy delivery devices employing a center fed dipole component (see, e.g., U.S. patent application Ser. No. 11/728,457; herein incorporated by reference in its entirety). The devices are not limited to particular configurations. In some embodiments, the devices have therein a center fed dipole for heating a tissue region through application of energy (e.g., microwave energy).

In some embodiments, the energy delivery systems of the present invention utilize imaging systems comprising imaging devices. The energy delivery systems are not limited to particular types of imaging devices (e.g., endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, electromagnetic imaging) (see, e.g., U.S. Pat. Nos. 6,817,976, 6,577,903, and 5,697,949, 5,603,697, and International Patent Application No. WO 06/005,579; each herein incorporated by reference in their entireties). In some embodiments, the systems utilize endoscopic cameras, imaging components, and/or navigation systems that permit or assist in placement, positioning, and/or monitoring of any of the items used with the energy systems of the present invention.

In some embodiments, the energy delivery systems provide software is configured for use of imaging equipment (e.g., CT, MM, ultrasound). In some embodiments, the imaging equipment software allows a user to make predictions based upon known thermodynamic and electrical properties of tissue, vasculature, and location of the antenna(s). In some embodiments, the imaging software allows the generation of a three-dimensional map of the location of a tissue region (e.g., tumor, arrhythmia), location of the antenna(s), and to generate a predicted map of the ablation zone.

In some embodiments, the energy delivery systems of the present invention utilize identification elements (e.g., RFID elements, identification rings (e.g., fidicials), barcodes, etc.) associated with one or more components of the system. In some embodiments, the identification element conveys information about a particular component of the system. The present invention is not limited by the information conveyed. In some embodiments, the information conveyed includes, but is not limited to, the type of component (e.g., manufacturer, size, energy rating, tissue configuration, etc.), whether the component has been used before (e.g., so as to ensure that non-sterile components are not used), the location of the component, patient-specific information and the like. In some embodiments, the information is read by a processor of the present invention. In some such embodiments, the processor configures other components of the system for use with, or for optimal use with, the component containing the identification element.

The energy delivery systems of the present invention are not limited to particular types of tracking devices. In some embodiments, GPS and GPS related devices are used. In some embodiments, RFID and RFID related devices are used. In some embodiments, barcodes are used.

In such embodiments, authorization (e.g., entry of a code, scanning of a barcode) prior to use of a device with an identification element is required prior to the use of such a device. In some embodiments, the information element identifies that a components has been used before and sends information to the processor to lock (e.g. block) use of system until a new, sterile component is provided.

The systems of the present invention are not limited to particular uses. Indeed, the endoscopy systems of the present invention are designed for use in any setting wherein, imaging, biopsy collection, or emission of energy is applicable. Such uses include any and all medical, veterinary, and research applications. In addition, the systems and devices of the present invention may be used in agricultural settings, manufacturing settings, mechanical settings, or any other application where energy is to be delivered.

In some embodiments, the systems are configured for open surgery, percutaneous, intravascular, intracardiac, endoscopic, intraluminal, laparoscopic, or surgical delivery of energy. In some embodiments, the energy delivery devices may be positioned within a patient's body through a catheter, through a surgically developed opening, and/or through a body orifice (e.g., mouth, ear, nose, eyes, vagina, penis, anus) (e.g., a N.O.T.E.S. procedure). In some embodiments, the systems are configured for delivery of energy to a target tissue or region. In some embodiments, a positioning plate is provided so as to improve percutaneous, intravascular, intracardiac, laparoscopic, and/or surgical delivery of energy with the energy delivery systems of the present invention. The present invention is not limited to a particular type and/or kind of positioning plate. In some embodiments, the positioning plate is designed to secure one or more energy delivery devices at a desired body region for percutaneous, intravascular, intracardiac, laparoscopic, and/or surgical delivery of energy. In some embodiments, the composition of the positioning plate is such that it is able to prevent exposure of the body region to undesired heat from the energy delivery system. In some embodiments, the plate provides guides for assisted positioning of energy delivery devices. The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, sterilization procedures (e.g., ablation of the fallopian tubes) and cauterization of blood vessels or tissue for any purposes. In some embodiments, the surgical application comprises ablation therapy (e.g., to achieve coagulative necrosis). In some embodiments, the surgical application comprises tumor ablation to target, for example, metastatic tumors. In some embodiments, the device is configured for movement and positioning, with minimal damage to the tissue or organism, at any desired location, including but not limited to, the lungs, brain, neck, chest, abdomen, and pelvis. In some embodiments, the systems are configured for guided delivery, for example, by computerized tomography, ultrasound, magnetic resonance imaging, fluoroscopy, and the like.

In some embodiments, the present invention provides systems that access to a difficult to reach region of the body (e.g. the periphery of the lungs). In some embodiments, the system navigates through a branched body structure (e.g. bronchial tree) to reach a target site. In some embodiments, systems, devices, and methods of the present invention provide delivery of energy (e.g. microwave energy, energy for tissue ablation) to difficult to reach regions of a body, organ, or tissue (e.g. the periphery of the lungs). In some embodiments, the system delivers energy (e.g. microwave energy, energy for tissue ablation) to a target site though a branched structure (e.g. bronchial tree). In some embodiments, the system delivers energy (e.g. microwave energy, energy for tissue ablation) to the periphery of the lungs through the bronchi (e.g. primary bronchi, secondary bronchi, tertiary bronchi, bronchioles, etc.). In some embodiments, accessing the lungs through the bronchi provides a precise and accurate approach while minimizing collateral damage to the lungs. Accessing the lung (e.g. lung periphery) from outside the lung requires puncturing or cutting the lung, which can be avoided by bronchial access. Insertion through the lung has medical complications that are avoided by the systems and methods of embodiments of the present invention.

All publications and patents mentioned in the above specification are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. An endoscopy directing device, comprising:
an endoscopy tool opening,
an endoscopy tool movement component, and
an endoscopy tool attachment component,
wherein the endoscopy tool movement component is positioned above the endoscopy tool attachment component,
wherein the endoscopy tool opening is a hollow channel that extends through the endoscopy tool movement component and the endoscopy tool attachment component,
wherein the endoscopy tool movement component is configured to incrementally move an endoscopy tool positioned within the endoscopy tool opening.

2. The endoscopy directing device of claim 1,
wherein the endoscopy tool attachment component is configured to secure with an endoscopy tool port.

3. The endoscopy directing device of claim 1,
wherein the width of the endoscopy tool opening is between 2 and 4 mm.

4. The endoscopy directing device of claim 1,
wherein the endoscopy tool movement component comprises two or more rotating wheels designed to simultaneously engage with an endoscopy tool positioned within the endoscopy tool opening such that rotation of such rotating wheels results in the incremental movement of the endoscopy tool.

5. The endoscopy directing device of claim 1,
wherein the rotation of the two or more rotating wheels is manual.

6. The endoscopy directing device of claim 1,
wherein the rotation of the two or more rotating wheels is automatic.

7. The endoscopy directing device of claim 1,
wherein the amount of incremental movement is between 1 and 2 mm.

8. The endoscopy directing device of claim 1,
wherein the endoscopy tool attachment component is configured to secure with an endoscopy tool port.

9. The endoscopy directing device of claim 1,
wherein the endoscopy tool is a microwave ablation device.

10. A system comprising:
a) the endoscopy directing device of claim 1; and
b) an endoscope, wherein said endoscopy tool attachment component is engaged with an endoscopy tool port of said endoscope.

11. The system of claim 10, wherein said endoscope is a bronchoscope.

12. The system of claim 10, wherein said system further comprises an endoscopy tool.

13. The system of claim 12, wherein said endoscopy tool is positioned in the endoscopy tool opening of said device.

14. The system of claim 12, wherein said endoscopy tool is selected from the group consisting of a biopsy tool and an ablation tool.

15. The system of claim 14, wherein said ablation tool is a microwave ablation device.

16. The system of claim 10, further comprising a processor for operation of the components of said system.

17. A method of directing an endoscopy tool, comprising:
a) providing an endoscopy tool, an endoscope, and an endoscope directing device of claim 1,
b) securing the endoscopy directing device with the endoscopy tool port,
c) positioning the endoscopy tool through the endoscopy tool opening such that the rotational wheels are in contact with the endoscopy tool,
d) directing the endoscopy tool to a preferred location through rotation of the rotational wheels.

18. The method of claim 17, wherein said endoscopy tool is located in a lung of a subject.

19. The method of claim 17, wherein the endoscopy tool is a microwave ablation device.
